(19) United States
(12) Patent Application Publication
(10) Pub. No.: US 2021/0218149 A1
(43) Pub. Date: Jul. 15, 2021

(54) Solution For Absorption Of Microwaves

(71) Applicant: MEDFIELD DIAGNOSTICS AB, Gothenburg (SE)

(72) Inventors: Hana Dobsicek Trefna, Rävlanda (SE); Anna Ström, Gothenburg (SE); Tiina Nypelö, Gothenburg (SE)

(73) Assignee: MEDFIELD DIAGNOSTICS AB, Gothenburg (SE)

(21) Appl. No.: 17/057,932

(22) PCT Filed: May 22, 2019
(86) PCT No.: PCT/EP2019/063248

§ 371 Date: Nov. 23, 2020

Related U.S. Application Data
(60) Provisional application No. 62/675,335, filed on May 23, 2018.

(51) Int. Cl. H01Q 17/00; A61B 5/0507; C08J 3/075; H01Q 1/52

(52) U.S. Cl. CPC H01Q 17/007; A61B 5/0507; C08J 3/075; C08J 2405/00; A61B 2562/182; C08J 2305/00; H01Q 1/525

(57) ABSTRACT

A microwave absorbing materials or gel is disclosed. In some examples, the material can be used in communication, radar, or industrial applications or to reduce the cross section of aircrafts or ships. The gel can be used as an absorbing material is to dampen microwaves. For example, to reduce coupling between antennas, shielding of measurement system to remove disturbances from internal or external error sources, provide a matching between the antenna and the body. The absorbing material can be manufactured as a flexible and moldable gel from polymers, water, and mineral salt. By altering the ratios of the content different damping and mechanical properties of the gel can be obtained. The surface of the material is modified to densify the surface and to reduce the surface slipperiness, avoid evaporation from the gel and to enable disinfection of the surface.

Example embodiments presented herein relate to a solution for absorption, (damping) of electromagnetic microwaves, and disclosure of a material, and a manufacturing process for such material.

### BACKGROUND

Microwave absorbers are used in several different systems and applications. The purpose is to attenuate unwanted fields and mitigate for example mutual coupling between antenna elements, dampen fields between cables or connectors or between parts of microwave systems, further to shield systems from external error sources, or to dampen field propagation in unwanted directions. Examples of application areas include communication, radar, ranging, industrial sensing, etc. Absorbers are also used to cover aircrafts, ships etc. that are designed to have stealth properties. In the last decades microwave systems have also been developed for medical diagnostic and treatment purposes. Examples include microwave tomography for breast cancer imaging and stroke and trauma diagnostics. Microwave systems have also been developed for treatment of cancer by applying focused or non-focused microwave fields in order to heat the tumor.

Other applications where the used of microwave absorbers are used include for example detection of tree health and quality, wood quality, moisture content of wood or products derived from wood, detection of defects in trees and wood, detection of dimensions, etc. in forest and wood industries. Yet other applications where microwave absorbers could prove to be useful include applications for detection of foreign and unwanted objects in food in food processing industries. Microwave absorbers can also be used in systems designed for detection, sensing and monitoring applications of flows, bulk material or others types of compounds in processing industries.

Microwaves typically refer to electromagnetic waves in the frequency range 0.1-30 GHz. While this frequency range may be the primary choice for the applications exemplified here, the use of microwave absorbers is not limited to that range. The embodiments relating to a microwave absorber described herein are thus not limited to this frequency range, but can also be used in applications operating at both higher and lower frequencies. In the rest of the document said signals will be referred to as radio frequency signals

Microwave systems with transmitting and/or receiving capability often consist of more than one antenna. Signal coupling directly between such antennas, without passing through the object of investigation, disturbs the measurements and is therefore unwanted. In some applications one can apply calibration procedures to compensate for this signal coupling, but calibration techniques are not always effective or useful. A better solution would be to physically isolate such parts from each other with shields or absorbers. Various solutions for absorbing microwave energy exist on the market, such as ferrites, sheets of absorbing foam and many other varieties. Metals or metal grids can be used for shielding in some cases, such as on the front screen of a microwave oven.

Common absorbers found on the market often consist of polyurethane foam that is treated with carbon in order to make the material conductive, and thereby attenuating. The conductive profile can be tailored to give the desired attenuating effect. While the material is somewhat flexible, it is not flexible enough for some applications. Usually, the more carbon is added, and thereby the more conductive the material, the less flexible it becomes. In some applications, it is not possible to obtain enough dampening while still maintaining sufficient flexibility and thereby allowing the absorber to fill out spaces and conform to bodies and surfaces. The dampening effect of the absorbers are also relatively weak, particularly at frequencies below 1 GHz, making it impossible to obtain enough dampening with a sufficiently thin absorbing foam in some applications. Other absorbers found on the market consists of ferrite powder mixed for example in a resin or rubber.

Therefore, there is a need to mitigate or solve the above issues.

### SUMMARY

An objective of embodiments herein is to obviate at least one of the above disadvantages with improved dampening in a material intended as a microwave absorber that is flexible and which can fill out spaces and cavities and conform to irregular surfaces.

One aspect of the embodiments described herein is a material that can be used as a dampening material. The material consists of a mixture of water and gel-forming polysaccharides or natural polymers. The material also contains a mineral salt to make it conductive and therefore to make it work as an attenuator of microwaves. The surface of the material is optionally modified to densify the surface and to reduce the surface slipperiness, avoid evaporation to increase the lifetime of the gel and to allow for disinfecting the surface with ethanol. Optionally a preservative can be added to stop microbiological growth.

Some of the advantages with the material is that by alternating the type and amount of natural polymer the texture of the gel can be altered. This is useful as properties can be optimized in order to best suit a particular application. Further, by changing the amount of salt the attenuating effect can be determined to suit the needs of a particular application.

Another advantage is that the material can be molded into a shape for perfect fitting in the target application.

Another advantage is that the ingredients in the material are low cost, readily available, renewable, non-toxic and regarded as commodity material in the food and pharmaceutical industry.

Low cost ingredients make the material particularly suitable for single use applications. One example being in health care applications where single use products are needed due to hygiene requirements.

### BRIEF DESCRIPTION OF THE DRAWINGS

FIG. 1. A schematic diagram illustrating an example diagnostics system.

FIG. 2. Schematically illustrates a system for detection.

FIG. 3. The gel.

FIG. 4. The surface coating as applied around the gel.

FIG. 5. The gel as applied around one antenna.

FIG. 6. The gel as applied around several antennas.

FIG. 7. The gel as applied on a surface to mitigate reflections.

FIG. 8. A typical operational situation where the gel is used to mitigate direct cross coupling between antennas.

FIG. 9. The gel is applied between an antenna and the surface to improve coupling of microwave signals.

FIG. 10. Results from measurements of transmission through an example gel.

FIG. 11. The gel comprising multiple layers of gel with different properties.

### DETAILED DESCRIPTION

A typical user scenario of the method for absorbing radio frequency signals using a gel according to what is disclosed herein is for detection and diagnostic applications relating to what is disclosed in patents, EP 2032030 B1, U.S. Pat. No. 9,332,922 B2, EP2020915B1, U.S. Pat. No. 8,724,864 B2.

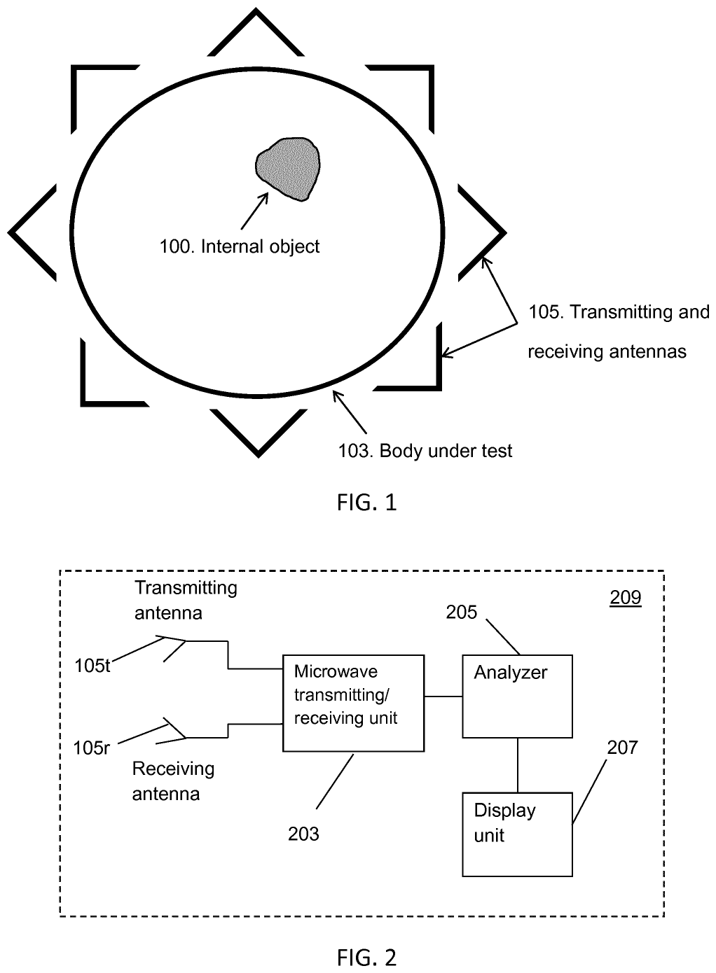

FIG. 1 illustrates a typical example of a system with one antenna for detecting an internal object 100 in a body under test 103. If only one antenna is used only reflection measurements can be made. The internal object 100 and the body under test 103 are not parts of the system. The body under test 103 may be a head, a brain, an abdomen, a thorax, a leg or any other body under test part of a human, an animal, or it may be any other form of biological tissue such as for example a tree or wood. The body under test 103 may also be non-living tissue, and of non-biological origin, such as, but not limited to plastics, etc. The body under test 103 may also be referred to as a dielectric medium, an object under investigation, a larger object, etc. The internal object 100 may also be referred to as an immersed object, a dielectric target, etc. The internal object 100 may be in the form of solid, semisolid, liquid or gas. The internal object 100 may be referred to as an immersed object in a larger object or body under test 103. The internal object 100 may also be referred to as a dielectric target, with certain properties, such as size, shape, position, dielectric parameters, etc. that is immersed inside another dielectric medium, i.e. the body under test 103. The internal object 100 may be a bleeding, a clot, an edema, a nail, a twig etc. Note that FIG. 1 only illustrates one internal object 100, but any number of internal objects 100 may be present in the body under test 103, including no objects at all. One internal object 100 is shown for the sake of simplicity.

Some components of the system described herein are depicted in FIG. 2 and consists of at least one transmitting antenna 105t and at least one receiving antenna 105r. When using the reference number 105 without the letters t or r, it refers to any of the transmitting and receiving antennas. It should be noted that these two antennas 105 may be combined in one antenna and in such case a directing mechanism (not shown) may be arranged in the path between the antenna 105 and the microwave transceiver inside the transceiver or as an external device. The combined transmitting and receiving antenna 105 may be referred to as a transceiver. The directing mechanism may be used in order to not transmit directly into a receiving unit in the transceiver possibly saturating the input electronics. A combination of the microwave transmitting/receiving unit 203 and the analyzer 205 may have a direction dependent component, e.g. the directing mechanism, which controls the transmitted and received signal in different directions. This may take place at the same time, i.e. transmission and reception may take place simultaneously. The directing mechanism may also be referred to as a switching mechanism. The transceiver may comprise two separate units, a transmitting unit and a receiving unit, or it may be built into one single unit with electronics for each function built into the single unit. The antennas are connected to a microwave transmitting/receiving unit 203 that is adapted to transmit and to receive radio frequency signals to and from the antennas 105. The system may further comprise an analyser 205 that is arranged to control a display unit 207. The display unit 207 is adapted to display the analysed result of the signals e.g. on a screen. Signal analysis may be performed at another location by sending, through a network connection or using storage devices, measured signals to another analysis device, e.g. a central server or central computational device for post analysis and/or for storing of measured signals in a central storage facility. This analysis device may be the same as the analyser 205 in FIG. 2 or a different analyser. The execution of the detection algorithm in order to produce a detection result based on the microwave signal measurements is performed on the analyser 205, the results are then presented on the display unit 207. The components 105, 203, 205, 207 in any combination or even alone may be referred to as a microwave system 209.

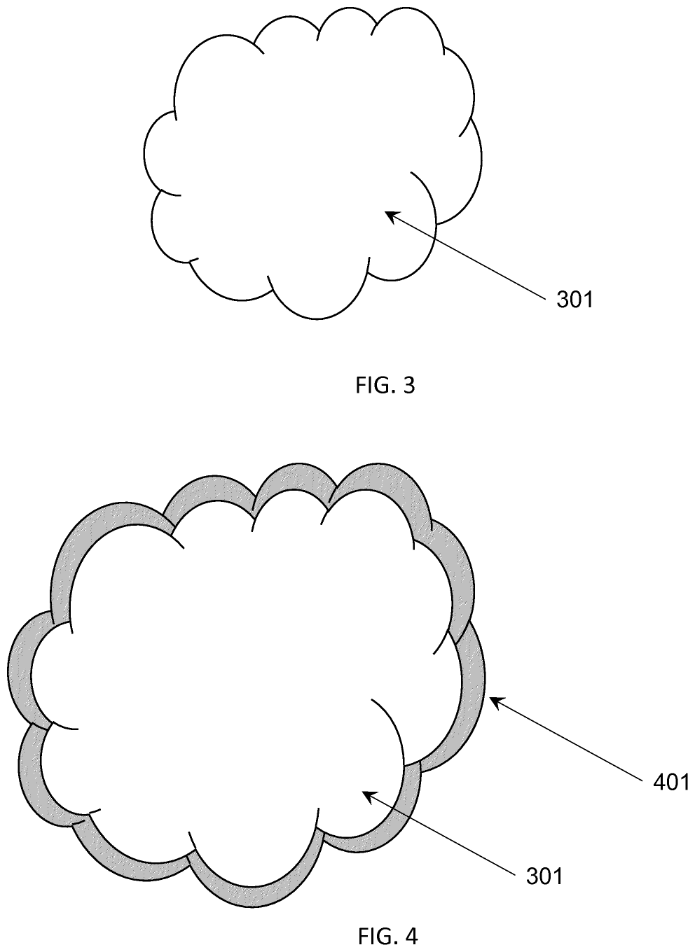

Some aspects disclosed herein relate to a material and manufacturing process of a material that is used as microwave absorber, that is flexible, and where the flexibility and absorbing capability can be altered. The manufacturing process of the material requires mixing of a liquid, natural polymers and mineral salt, when needed, and often also heating for the substances to mix. Upon cooling, the material forms a flexible gel 301 as illustrated in FIG. 3. The material is therefore ideally suited to be molded into desired shapes that are suitable for different applications. The flexibility of the gel 301 can also be utilized when the material is pressed onto a surface and the gel is to conform to the surface.

The material consists of a mixture of natural polymers, water, and mineral salt. In some examples where a low damping or no damping is wanted the mineral salt can be excluded, Different preservatives can be added in order to increase the life time of the gel 301 and avoid bacterial and microbial growth. The preservative could for example be different kind of benzoates or other substances used in the food industry, such as benzoic acid, nitrates or nitrites. Optionally the surface of the gel can be to prevent water evaporation and for ease of handling. This surface treatment can be made either chemically or with for example a plastic cover to completely mitigate dehydration of the gel.

Ingredients of the Gel

Natural polymers are used as ingredients of the gel and to make it thicken into a gel 301. Typical polymers are Xanthan with a galactoglucomannan. Agar and/or agarose could be used if a harder gel 301 is desired. Mineral salt is used to alter the conductive properties of the gel 301.

Xanthan gum is composed of a (1-4)-linked β-D-glucose backbone with a trisaccharide chain on every other glucose at C-3. Xanthan gum is an extracellular polysaccharide secreted by the bacterium Xanthomonas campestris.

A typical choice of galactoglucomannan is locust bean gum (LBG).

Agar or agarose extracted from red seaweed and composed of repetitive units of D-galactose and 3,6 anhydro-L-galactose.

Several different mineral salts can be used. Typical choices are NaCl and CaCl and typical concentrations are 1-15 weight %.

Manufacturing of the Gel

Gel 301 preparation: All polymers are slowly added to deionized water at room temperature under vigorous stirring. The solution is heated to 95° C. and kept (typically for 30 minutes) at this temperature until the polymers are fully dissolved. The gels can be formed from the solution, for example by molding, into the desired shape. During cooling gelation occurs. By varying the relative amount of ingredients the properties of the gel 301 can be tuned.

For the purpose of demonstration, different example gels 301 were prepared. Properties of the different gels 301 were assessed in order to illustrate the characteristics of the gels 301. The ingredients used in Example 1 is: Xanthan with the Galactoglucomannan LBG. Gels 301 with different concentration of salt were prepared. In Example 2: Xanthan and Galactoglucomannan and Agar/Agarose. Gels 301 with different concentration of salt were prepared.

Example 1: Gel based on Xanthan/LBG. Xanthan/LBG gels 301 are obtained by mixing Xanthan and LBG dispersions at a 1:1 ratio at 95° C., salt is added and the mixtures are poured into molds and let to cool upon which gelation occurs. Total polymer concentration ended at 1%. Different gels 301 with varying salt (NaCl) concentration, 0%, 1%, 4% were prepared.

Example 2: Xanthan/LBG and Agarose gels 301 are prepared similarly as in Example 1, but Agarose is added to the already mixed Xanthan/LBG dispersion at 90° C. While the final polymer concentration is kept at 1% and the ratio of Xanthan/LBG is 1:1, the ratio between the difference polymers vary. In this example the ratio of polymers in the Xanthan/LBG/Agarose sample is 0.4:0.4:0.2. In this case salt (NaCl) concentrations of 0% and 1% were used.

Example gel 1 301 and Example gel 2 301 were evaluated according to large deformation (stress relaxation), gel strengths (small deformation oscillatory rheology) and dielectric properties. The resulting properties are shown in table 1. The findings as illustrated in the table are summarized here: Stress relaxation of the gel 301 describes the behavior of the gel 301 under stress over a specific period of time. The value of Fₘₐₓ correspond to the max force required to actually compress with 30% and Fᵣₑₗₐₓ, the force required to maintain the deformation at the end of the 300 sec. The results show that a) the amount of added salt does not change the general strength nor the behavior of the gel 301 over the time tested, b) the relaxation of the material is low, that is, the material does not appear to rearrange substantially during the compression and c) no cracks or damages of the material were observed at 30% deformation. The onset of gelation (T_{g}) is here defined to be the temperature at which the storage modulus (G′) increases rapidly and is higher than the loss modulus (G″). The dispersion was loaded to the rheometer hot and let to gel 301 on the rheometer as the temperature was reduced from 75° C. with 2° C./min. T_{g}, defined as above, was only slightly increased by the addition of salt. The melting temperature, Tₘ, representing the temperature at which G″ is larger than G′ upon heating of the gel 301, shows that also Tₘ is largely unaltered by the presence of salt. Furthermore, the exact match of T_{g} and Tₘ of the Xanthan/LBG gel 301 is expected. The addition of agarose to the system increases the strength of the gel 301 and also the Tₘ, in line with expectation based on the properties of pure agarose gels showing thermal hysteresis. In general, the mechanical and rheological measurements of the gels 301 showed that the addition of salt did not influence such properties of the gels 301. The results further show some general properties such as behavior upon stress, gelation and melting temperature and gel strengths.

TABLE 1
Table of characteristics of the two example gels 301
with different salt concentration, and where Fₘₐₓ and
Fᵣₑₗₐₓ are obtained from stress relaxation tests, G′, G″,
T_{g}, Tₘ are obtained from oscillatory measurements.

| | Example 1: | | | Example 2: | |
|---|---|---|---|---|---|
| | Xanthan/LBG | | | Xanthan/LBG/agarose | |
| | Added NaCl | | | Added NaCl | |
| Gel | 0% | 1% | 4% | 0 | 1% |
| Fₘₐₓ/N | 0.12 | 0.12 | 0.12 | — | — |
| Fᵣₑₗₐₓ/N | 0.08 | 0.08 | 0.08 | — | — |
| G′ at T = 20° C./Pa | 200 | 200 | 200 | 300 | 400 |
| T_{g} ° C. | 58 | 62 | 62 | 60 | 60 |
| Tₘ ° C. | 58 | 62 | 62 | >80 | >80 |
| Electrical conduc- | 0.3 | 1.8 | 3.15 | 0.3 | 2.0 |
| tivity (at 1 GHz) | | | | | |
| [S/m] | | | | | |

The characterization of the gel 301 as illustrated in Table 1 was made with the following methods:

The stress relaxation of the gels were tested using a Texture Analyser (HDi, Stable Micro Systems, UK) equipped with a 5 kg load cell. Compression tests were performed with a cylindrical probe of 1 cm diameter. The gels tested were of 1 cm in diameter and a height of 1 cm. The stress required to maintain the gel at 30% strain was tested over 300 sec. The oscillation tests were done using a DHR-3 from TA instruments. The geometry used was a cone and plate with a diameter of 40 mm and a gap of 20 μm. The rheological properties of the gels were tested at a frequency of 1 Hz and a strain of 0.5%. The temperature was controlled by a Peltier plate. All samples were loaded at high temperatures, that is in their liquid state, and let to gel 301 on the rheometer. A solvent trap from TA instruments was used to reduce evaporation. The conductivity was measured with a Keysight dielectric probe, 85070E.

Surface Coating

Surface modification: A typical user scenario is shown in FIG. 4 where the surface of a gel 301 is modified by applying a coating 401 on the gel. The coatings 401 reduce evaporation of water thus avoids dehydration of the gel 301. This increases the life-time of the gel. The coating 401 also reduces slipperiness of the surface. It is possible to use a multilayer coating. One advantage with the surface coating is that for example ethanol can be used to rinse the surface after coating 401 in order to disinfect the surface and to reduce microbial growth.

Surface coating alternative 1: The gel 301 surface is coated 401 with water-solution/dispersion of natural polysaccharides such as starches. The polysaccharides then attach to the gel surface and provide densification creating a continuous coating on the surface.

Surface coating alternative 2: The gel 301 surface is coated 401 with alternating layers of cationic and anionic molecules. For example, cationic starch is used as the first layer for forming a continuous film surface. Anionic montmorillonite particles are adsorbed on the starch layer providing barrier against water transport out of the gel (evaporation).

Surface coating alternative 3: The gel 301 is coated 401 with a plastic material. The plastic can be thin, thick, flexible or rigid depending on the desired properties of the sealing,

User Scenarios of Gel 301

In FIG. 3 a typical user scenario of the gel 301 is shown. The gel is configured to a particular spatial location. The purpose with the gel is to alter the electromagnetic properties, i.e. the dielectric properties permittivity, conductivity and permeability, in the domain where the gel is located. Consequently, FIG. 3 illustrates a modification of one or more electromagnetic properties of an environment in which a microwave electromagnetic field propagates by arranging the gel at a location in the environment such that microwave signals propagating in a vicinity of the location interacts with the gel.

In FIG. 4 a typical user scenario is shown where the gel 301 is sealed with a coating layer 401.

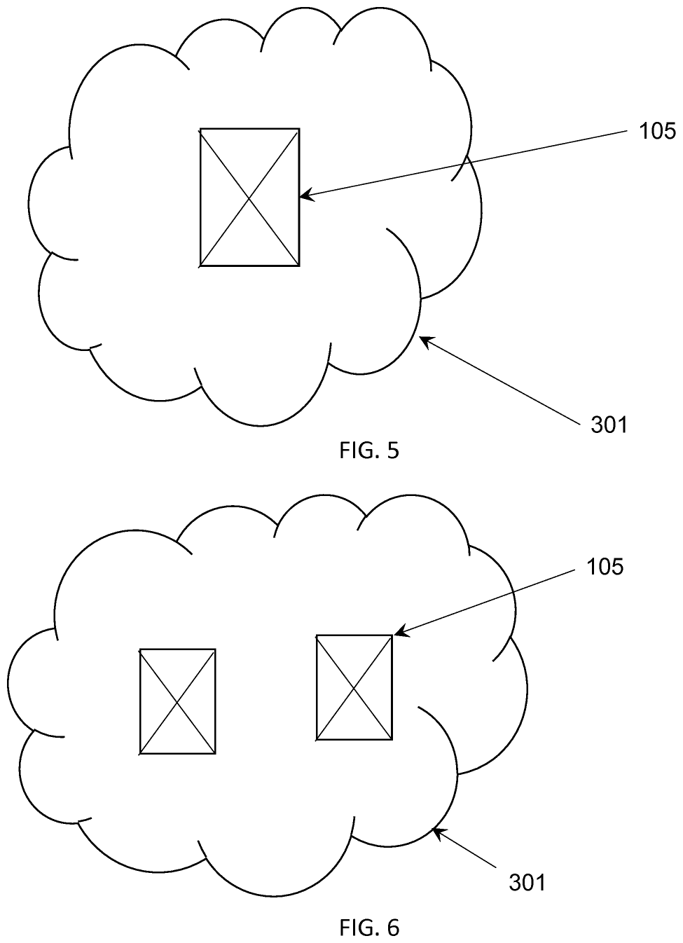

In FIG. 5 a typical operational scenario is shown. In the figure one antenna 105 is shown. The antenna can be used either as a transmitter or as a receiver, or both. The antenna is immersed or surrounded by the gel 301. The gel 301 can be applied on the sides, at the back or in front of the antenna 105, depending on which direction transmitting or receiving signals are to be attenuated.

In FIG. 6 a different user scenario is shown. Two antennas 105, are shown where the antennas are immersed, or surrounded by the gel 301. Gel 301 present between antennas 105 are intended to mitigate direct coupling between antennas, and instead ensure the transmitting or receiving directions of microwaves are in any other direction and in accordance with the desired application. For a person, skilled in the art this operation is denoted as reduction of direct mutual coupling between antennas. Direct signals are usually a source of disturbances and therefore unwanted, the strength of the direct signal is often very large in comparison to the signal of the sources related to the intended application. In such application, it could be an advantage to mold the gel 301 to fit perfectly to the antennas 105.

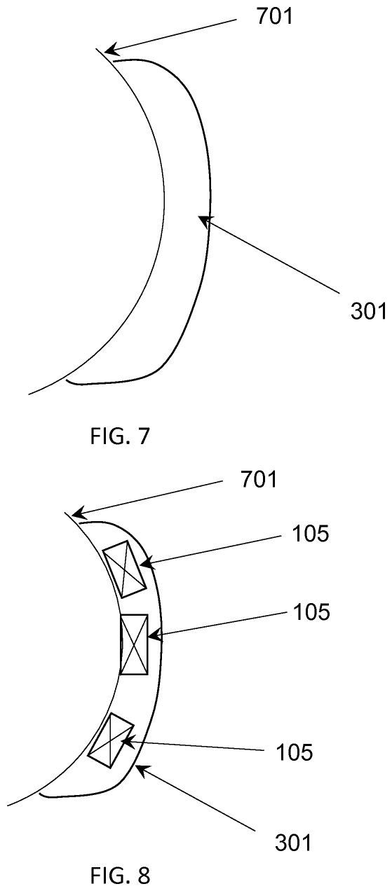

FIG. 7. shows a user scenario where the gel 301 is applied onto a surface 701. The surface 501 can consist of any material. In this configuration, the intended operation is to lower the reflection of microwaves from the surface 701. Incoming waves propagate from the surroundings, into the gel 301, reflects from the surface 701 and attenuate also on the way back through the gel 301 such that the reflected signals are partially or completely attenuated. A typical use is to lower the radar cross section of an object by covering its surface 701 with the gel 301. The object could for example be an aircraft that is designed to have stealth properties, or an antenna supporting structure which interaction with the antenna should be minimized.

FIG. 8. In this user scenario three antennas 105 are applied onto a surface 701. The application could for example be to conduct sub surface 701 sensing using radio frequency signals that are transmitted and received from the antennas 105. In this application, the desire is to transmit signals from the antennas 105 through the surface 701. Objects below the surface 301 could give rise to scattered signals that propagate back out from the surface 301 again to be detected with the antennas 301. Direct coupling, i.e. signals that propagate between the antennas directly are unwanted as they introduce disturbing signals in the measurements. Different calibration procedures are sometime utilized in an attempt to remove such direct signals. But a better option it to attenuate them completely or partially. The embodiments described herein are particularly useful when the antennas are to be pressed against the surface and the flexibility of the gel allows good fitting to the surface.

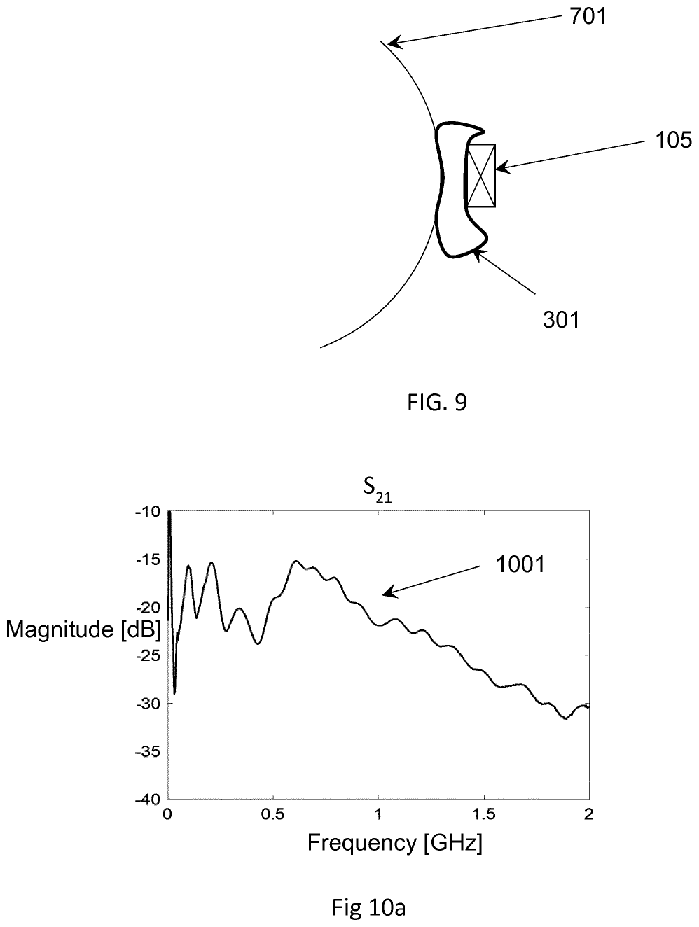

FIG. 9. In this user scenario the gel 301 is applied between the antenna 105 and the surface 701. This is a scenario which could be useful for applications of sub surface 701 sensing. The purpose of the gel in this case is to constitute a matching medium between the antenna 105 and the surface 701 and thereby to ensure better coupling of energy past the surface 701 and into the object. The properties of the gel can then be impedance matched to the antennas and the body to optimize the transfer of energy past the interfaces of different materials. An advantage here is that the gel can conform both to the antenna 105 itself, and the surface 701. In that way for example air gaps between the antenna 105 and surface 701 can be avoided and thus resulting in better matching than if the antenna 105 itself is applied directly on the surface 701.

Transmission Measurements of an Example Gel

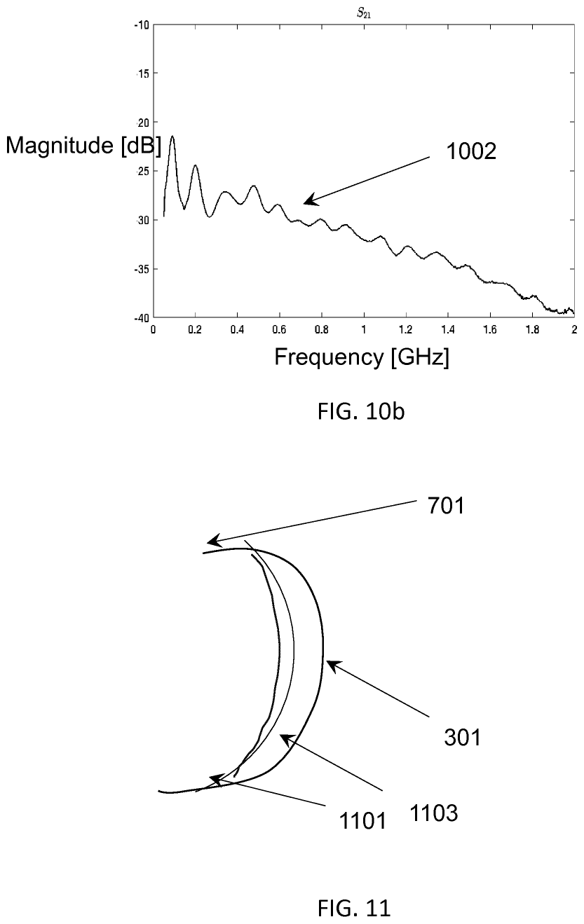

An example gel, according to the recipe in Example 1 and containing 0.8% and 5% NaCl respectively. The first gel was 18 mm thick and the second gel was 25 mm thick. In FIG. 10. transmission measurements are shown, using two patch antennas placed on opposite sides of the gel and in direct contact with the gel. FIG. 10a show transmission through the 18 mm thick gel with 0.8% NaCl 1001, and in FIG. 10b the transmission of the 25 mm thick gel with 5% NaCl, 1002.

There has been disclosed herein techniques and materials for absorption of electromagnetic microwaves, and a manufacturing process for such material, as summarized by the below list of numbered embodiments.

1. A solution for attenuating microwave signals consisting of a material and a manufacturing procedure of an absorbing material. The manufacturing procedure resulting in a gel that can be used as an attenuator of signals in microwave applications. The said gel comprises at least one polysaccharide or natural polymer and a metal salt/s that is mixed in ratios appropriate for obtaining the desired properties.

2. A solution for attenuating microwave signals consisting of a material and a manufacturing procedure of an absorbing material as of claim 1 used in applications
with the purpose to reduce the interference of signals between (microwave) antennas whereby the gel is placed between antennas,
for damping of external error sources, whereby the gel is placed such that it surrounds the antenna,
for damping of internal and unwanted coupling between different internal parts of the microwave system, whereby the gel is placed between or such that it surrounds the parts of the system that are to be shielded from each other
as a matching medium, whereby the gel is placed between the antenna and the surface,
for reducing the scattering from a surface, whereby the surface is covered with the gel.

3. A solution for attenuating microwave signals consisting of a material and a manufacturing procedure of an absorbing material of any of the claims 1-2 where the gel is molded in the presence of the antennas in order to ensure perfect fitting of the gel to the antenna.

4. A solution for attenuating microwave signals consisting of a material and a manufacturing procedure of an absorbing material of any of the claims 1-3 where the gel is manufactured and used as a single use product, where the product is adapted for easy mounting and dismounting from the set of antennas.

5. A solution for attenuating microwave signals consisting of a material and a manufacturing procedure of an absorbing material of the claims 1-4 where the surface of the gel is modified and thereby sealed, for the purpose of reducing evaporation.

6. A solution for attenuating microwave signals consisting of a material and a manufacturing procedure of an absorbing material of the claims 1-4 where the surface of the gel is modified and thereby sealed, for the purpose of reducing slipperiness.

7. A solution for attenuating microwave signals consisting of a material and a manufacturing procedure of an absorbing material of the claims 1-4 where the surface of the gel is modified such to enable disinfecting the surface.

8. A solution for attenuating microwave signals consisting of a material and a manufacturing procedure of an absorbing material of the claims 1-7 where the natural polymers xanthan, galactoglucomannan, agar and agarose are used in any proportion to produce the gel.

9. A solution for attenuating microwave signals consisting of a material and a manufacturing procedure of an absorbing material of the claims 1-8 where the surface of the gel is coated by applying a water-solution/dispersion of natural polysaccharides on the surface.

10. A solution for attenuating microwave signals consisting of a material and a manufacturing procedure of an absorbing material of the claims 1-8 where the surface of the gel is coated with alternating layers of cationic and anionic molecules.

11. A solution for attenuating microwave signals consisting of a material and a manufacturing procedure of an absorbing material of the claims 1-10 where different gels with different ingredients and/or salt concentration are stacked on top of each other to form a attenuating gel with gradually changing properties.

Detailed Description of User Scenarios for the Disclosed Gel 301

Here we describe how the techniques and materials for absorption of electromagnetic radio frequency signals, and a manufacturing process for such material may be utilized in a method for attenuating radio frequency signals. The method modifies one or more electromagnetic properties of an environment in which a microwave electromagnetic field propagates by arranging the gel at a location in the environment such that microwave signals propagating in a vicinity of the location interacts with the gel.

The manufacturing process revealed herein discloses how a gel 301 can be manufactured and used as described below, to absorb radio frequency signals. There are various other absorbers available on the market. Some advantages are that the gel 301 is made of very inexpensive ingredients, its constituents are basically ingredients from the food industry and therefore also non-poisonous and harmless. As compared to other gels 301 on the market that contains carbon powder, ferrites or other materials which might be more problematic if in contact with skin. These materials are also not so easily molded directly in situ of a microwave system 209 as the manufacturing process is often more involved,

M1. A method for attenuating microwave signals,
(i) where the attenuation is obtained by using an absorbing material in form of a gel 301,
(ii) where the gel 301 is configured to occupy domains such that radio frequency signals entering into that domain are absorbed and correspondingly attenuated,
(iii) where the said gel 301 comprises at least one polysaccharide or natural polymer and a metal salt mixed with water in any ratios.

The main advantage with using a gel 301 is that it can be molded or shaped in other ways to conform to different shapes. It is also flexible and therefore easily conforms to antennas 105 surfaces 701 and structures by applying pressure. This is an important property as it makes it easy to avoid for example pockets of air that otherwise might corrupt the measurements. Coupling between parts of the microwave system 209, for example direct coupling between antennas 105, cables or other parts of the system 209 might be very large, and result in large measurement errors. This coupling must be dampened, for example with said gel 301, as to ensure highest possible accuracy in the measurements. This is described in M2.

M2. The method as described in M1, comprising shaping the gel 301 and configuring said gel 301 to conform to parts of a microwave system 209 in order to reduce coupling of radio frequency signals between said parts within the system 209.

External sources of radio frequency signals, for example from communication systems such as cell phones of WIFI might operate on the same frequencies as a microwave system 209. These signals might corrupt the measurements. By covering parts of the microwave system 209 with gel 301 such signals can be attenuated, as described below in M3.

M3. The method as described in M1 comprising shaping the gel 301 and configuring said gel 301 to conform to parts of a microwave system 209 in order to reduce interfering radio frequency signals from external microwave emitters.

It is not uncommon that for example aircrafts or ships are designed as to have stealth properties, i.e. to be invisible to radar. Part of such design include covering surfaces 701 with absorbing material that attenuates incoming microwave signals. In certain applications it could be interesting to use the gel 301 described herein for such purposes. Solutions with either single layer gels 301 or gels with several layers, 1101, 1103 could be used.

M4. The method as described in M1, comprising shaping the gel 301 to conform to a surface 701 of a reflective material, whereby the reflectivity of the surface is lowered, whereby radio frequency signals incident on said surface 701 will be attenuated, whereby the reflectivity will be lowered.

A gel 301 can also be used to attenuate signals that are emitted from an antenna 105 before it is transmitted into a material, past a surface 701. In that case the properties and/or the thickness of the gel 301 could be adjusted to reach the desired power level of the transmitted signal.

M5. The method in any of the previous points, comprising configuring the gel 301 to conform to microwave emitters 105t and microwave receivers 105r in a microwave system 209, whereby the gel 301 acts to attenuate radio frequency signals as they enter into said gel 301.

The description below discloses how a microwave system 209, configured to make measurements of a body under test 103, further the surface 701 of the body under test 103 and the antennas 105 are covered with a gel 301 as shown in FIG. 8 in order to dampen signals from external microwave sources, as described in M3.

M6. The method as described in any of the previous points M1-M5, applied in a system 209 where the emitters 105t and receivers 105r comprise antennas 105, further comprising configuring the antennas 105 in proximity of a body under test 103. The method further comprising emitting radio frequency signals from the transmitting antenna 105t whereby said radio frequency signals propagate through the object 103 and detecting said signals with the receiving antenna 105r. The method further comprising configuring the gel 301 on the outside of the antennas 105 seen from the surface 701 of the object under test 103 whereby the gel 301 acts to attenuate unwanted coupling of signals between different internal parts of the microwave system 209 and the antennas 105 and from interference signals from external error sources are attenuated

The description below discloses how a microwave system 209, configured to make transmission measurements of a body under test where the gel 301 is applied between antennas 105, outside antennas 105 and on the surface 701 of the body under test 103. The result is that signals propagating directly between antennas 105, without first having propagated through the body under test 103 are dampened. Such direct signals are unwanted and usually leads to disturbances in the measurement data. In the literature one can find examples where such direct signals are compensated for by different calibration techniques. Such techniques are more effective when the object is located far away from the antennas 105, i.e. the far field, and where the antennas 105 are mounted in a rigid fixture such that the antenna 105 positions are fixed. In examples where the antenna 105 positions are flexible and in close proximity of the body under test 103 it is more effective to dampen such direct signals.

M7. The method as described in any of the previous points M1-M5, applied in a microwave system 209 where the emitters 105t and receivers 105r comprise antennas 105 comprising configuring the antennas 105 in proximity of a body under test 103. The method further comprising emitting radio frequency signals from the transmitting antenna 105t whereby said radio frequency signal propagate through the body under test 103 detecting said signals with the receiving antenna 105r. The method, as shown in FIG. 8, further comprising configuring the gel 301 between the transmitting 105t and the receiving antenna 105r whereby the gel acts to attenuate the interference signals propagating between the antennas outside the object.

The gel 301 can also be used between the antennas 105 and the surface 701 of the body under test 103 for the purpose of impedance matching. The purpose is to then to ensure that as much as possible of the radio frequency signals are propagating through the surface 701 of the body under test 103 and thereby ensuring that the signal strengths of the signal is propagating through the object is maximized.

M8. The method in point M1 applied in a system where the emitters 105t and receivers 105r comprise antennas 105 comprising configuring the antennas 105 in proximity of an body under test 103. The method further comprising emitting radio frequency signals from the transmitting antenna 105t whereby said microwave signal propagate through the body under test and detecting said signals with the receiving antenna 105r. The method, as shown in FIG. 9, further comprising configuring the gel 301 between the surface 701 of the body under test 103 and the antenna 105, further comprising selecting the properties of the gel 301 such that is acts as an impedance matching medium.

The mixture from which the gel 301 is manufactured is a liquid when it is heated, and when it is cooled the gel 301 is formed. Molding is therefore a convenient method of crafting the gel 301 into desired shape.

M9. The method in any of previous points, M1-M8 where the gel 301 is molded or in other ways manufactured to ensure close fitting between the gel and parts of the microwave system 209.

As the ingredients of the gel 301 are inexpensive, the gel 301 is particularly suited as a single use product. This might be for purposes of hygiene in health care, and also in cases where gels of different properties are needed in particular measurement situations.

M10. The method in any of the previous points, M1-M9, where the gel 301 is used as a single use product, where the product is adapted for easy mounting and dismounting to parts of the microwave system 209.

The gel 301 is manufactured of ingredients known from the food processing industry.

M11. The method in any of the previous points M1-M10 comprising the natural polymers xanthan, galactoglucomannan, agar and/or agarose in any proportion to produce the gel 301.

The gel itself will over time dry out as the water content evaporates, different methods of sealing 401 the surface of the gel as to avoid evaporation and maintain the initial water content and gel 301 properties over a prolonged duration as compared to when no surface coating is used. This will increase the life-time of the gel.

M12. The method in any of the previous points M1-M11, where the surface of the gel 301 is modified to be sealed 401 with the purpose of reducing evaporation of water from the gel in order to prolong the life time of the gel 301.

A plain gel is quite slippery due to the water bound in the gel 301. To simplify handling of the gel it is convenient to chemically modify or treat the surface 401 in order to make it less slippery.

M13. The method in any of the previous points M1-M12, where the surface of the gel 301 is modified and thereby sealed 401, for the purpose of reducing slipperiness.

Particularly in health care applications bacterial growth, growth of fungi or mould are unwanted. Chemically modifying or sealing 401 the surface as to simplify disinfection is described below.

M14. The method in any of the previous points M1-M13, where the surface of the gel is modified and thereby sealed such to enable disinfecting the surface.

The point below discloses some particular substances that could be used to treat the surface of the gel 301 as to accomplish what is described in any if the points M12-M14

M15. The method in any of the previous points M1-M14, where the surface of the gel 103 is coated 401 by applying a water-solution/dispersion of natural polysaccharides on the surface.

The point below discloses some particular substances that could be used to treat the surface of the gel 301 as to accomplish what is described in any if the points M12-M14

M16. The method in any of the previous points M1-M14 where the surface of the gel 301 is coated 401 with alternating layers of cationic and anionic molecules.

A method of sealing 401 the gel 103 is to wrap it in a plastic material. The plastic can be thin, thick, flexible or more rigid depending on the desired characteristics.

M17. The method in any of the previous points M1-M14 where the surface of the gel 103 is coated 401 with a layer of plastic.

There are applications where it is not enough to use one single gel 301 with a certain thickness and property, e.g. salt concentration, to achieve the desired damping properties. One alternative is therefore to manufacture a layered gel 301, of at least two different gels, constituting a layer 1101 stacked on another layer 1103 of different properties. The layered gel 301 can be made of an arbitrary number of layers of different properties or thickness.

M18. The method in any of the previous points M1-M17, where gels 301 comprising different amounts of ingredients and/or salt concentration, are configured in a layered structure to form a gel with gradually changing properties, such that a gel 1101 of certain properties are configured with another layer 1103 stacked on said gel 301.

To summarize the above discussions, there has been disclosed herein;

A method for modifying one or more electromagnetic properties of an environment in which a microwave electromagnetic field propagates, comprising providing a gel comprising at least one polysaccharide or natural polymer and

a metal salt mixed with water;

arranging the gel at a location in the environment such that microwave signals propagating in a vicinity of the location interacts with the gel.

The method optionally comprises arranging the gel at the location such that microwave signals propagating in a vicinity of the location are absorbed by the gel and thus the microwave electromagnetic field is modified so as to be attenuated.

Optionally, the location is adjacent to a part of a microwave system, the method comprising configuring a shape of the gel to conform to the part of the microwave system, thereby reducing coupling of microwave signals involving the part of the microwave system and/or attenuating an interfering microwave signal from an interfering microwave transmitter received by the part of the microwave system.

Optionally, the part of the microwave system corresponds to any of a microwave emitter, a microwave receiver, a microwave transceiver, and a microwave antenna.

Optionally, the location is adjacent to a radio frequency reflective surface, the method comprising configuring a shape of the gel to conform to the radio frequency reflective surface, thereby reducing a reflectivity property of the reflective surface.

Optionally, the location is in-between a microwave transmit antenna and a microwave receive antenna constituting parts of a microwave system, the method comprising configuring the antennas in proximity to an object of interest, and emitting a microwave signal from the transmitting antenna, whereby the transmitted microwave signal propagates through the object of interest and is received by the microwave receive antenna, whereby the gel attenuates unwanted coupling of signals propagating between the transmit and the receive antennas outside of the object of interest and/or attenuates an interfering microwave signal from an interfering microwave transmitter.

The method optionally comprises comprising arranging the gel at the location such that the gel acts as an impedance matching medium for microwave signals propagating in a vicinity of the location and thus the microwave electromagnetic field is modified in terms of impedance matching.

There was also discussed a gel for modifying one or more electromagnetic properties of a microwave electromagnetic field, comprising at least one polysaccharide or natural polymer and a metal salt mixed with water.

Optionally, the natural polymer comprises any of Xanthan, Galactoglucomannan, Agar and Agarose.

Optionally, a surface of the gel is treated or sealed to prevent evaporation and/or to increase friction and/or to allow for application of a disinfectant to the surface of the gel.

Optionally, the surface of the gel is sealed by any of application of a water-solution/dispersion of natural polysaccharides on the surface, application of a coating with alternating layers of cationic and anionic molecules, application of a layer of plastic on the surface of the gel.

Optionally, the gel is arranged with a layered structure, where each layer comprises a respective amount of ingredients and/or a respective salt concentration, thereby gradually altering electromagnetic properties of the gel.

There was also discussed use of the gel according to the above disclosure for attenuating a microwave signal, or for providing impedance matching with respect to an object of interest.

There was also discussed herein a microwave measurement system comprising at least a first and a second microwave antenna arranged to measure one or more properties of an object of interest by transmission and reception of microwave signals through the object of interest, wherein the microwave measurement system further comprises the gel according to the above disclosure, wherein the gel is arranged to reduce a coupling between at least the first and the second antenna.

There was also discussed herein a microwave measurement system comprising at least a first and a second microwave antenna arranged to measure one or more properties of an object of interest by transmission and reception of microwave signals through the object of interest, wherein the microwave measurement system further comprises the gel according to the above disclosure, wherein the gel is arranged to improve an impedance matching between any of the first and second antenna and the object of interest.

1. A method for modifying one or more electromagnetic properties of an environment in which a microwave electromagnetic field propagates, comprising:
providing a gel, the gel comprising a metal salt mixed with water and at least one polysaccharide or natural polymer;
arranging the gel at a location in the environment such that microwave signals propagating in a vicinity of the location interacts with the gel.

2. The method of claim 1, comprising arranging the gel at the location such that microwave signals propagating in a vicinity of the location are absorbed by the gel and thus the microwave electromagnetic field is modified so as to be attenuated.

3. The method of claim 2, wherein the location is adjacent to a part of a microwave system, the method comprising configuring a shape of the gel to conform to the part of the microwave system, thereby reducing coupling of microwave signals involving the part of the microwave system and/or attenuating an interfering microwave signal from an interfering microwave transmitter received by the part of the microwave system.

4. The method of claim 3, wherein the part of the microwave system is any one of: (i) a microwave emitter, (ii) a microwave receiver, (iii) a microwave transceiver, and (iv) a microwave antenna.

5. The method of claim 1, wherein the location is adjacent to a radio frequency reflective surface, the method comprising configuring a shape of the gel to conform to the radio frequency reflective surface, thereby reducing a reflectivity property of the reflective surface.

6. The method of claim 1, comprising:
configuring a microwave transmit antenna and a microwave receive antenna antennas in proximity to an object of interest,
emitting a microwave signal from the microwave transmit antenna, and
wherein:
the location is in-between the microwave transmit antenna and the microwave receive antenna constituting parts of a microwave system,
the transmitted microwave signal propagates through the object of interest and is received by the microwave receive antenna, and
the gel is configured to attenuates unwanted coupling of signals propagating between the microwave transmit antenna and the microwave receive antennas outside of the object of interest or the gel is configured to attenuates an interfering microwave signal from an interfering microwave transmitter.

7. The method of claim 1, comprising arranging the gel at the location such that the gel acts as an impedance matching medium for microwave signals propagating in a vicinity of the location and thus the microwave electromagnetic field is modified in terms of impedance matching.

8. A gel for modifying one or more electromagnetic properties of a microwave electromagnetic field, comprising at least one polysaccharide or natural polymer and a metal salt mixed with water.

9. The gel of claim 8, wherein the natural polymer comprises any of Xanthan, Galactoglucomannan, Agar and Agarose.

10. The gel of claim 8, wherein a surface of the gel is treated or sealed to prevent evaporation and/or to increase friction and/or to allow for application of a disinfectant to the surface of the gel.

11. The gel of claim 10, wherein the surface of the gel is sealed by any of application of a water-solution or dispersion of natural polysaccharides on the surface, application of a coating with alternating layers of cationic and anionic molecules, application of a layer of plastic on the surface of the gel.

12. The gel of claim 8, wherein the gel is arranged with a layered structure, where each layer comprises a respective amount of ingredients or a respective salt concentration, thereby altering electromagnetic properties of the gel layer by layer.

13. The gel of claim 8, wherein the gel functions to attenuate a microwave signal, or to provide impedance matching with respect to an object of interest.

14. A microwave measurement system comprising:
a first microwave antenna and a second microwave antenna arranged to measure one or more properties of an object of interest by transmission and reception of microwave signals through the object of interest,
a gel, the gel configured to modify one or more electromagnetic properties of a microwave electromagnetic field, and
wherein the gel is arranged to reduce a coupling between at least the first microwave antenna and the second microwave antenna.

15. A microwave measurement system of claim 14 wherein the gel is arranged to improve an impedance matching between either the first and second antenna and the object of interest.

16. The microwave system of claim 14 wherein the gel comprises a metal salt mixed with water and at least one of a polysaccharide or a natural polymer.

17. The gel of claim 12, wherein the gel functions to attenuate a microwave signal, or to provide impedance matching with respect to an object of interest.

18. The microwave measurement system of claim 15 wherein a surface of the gel is treated or sealed to do at least one of: (i) prevent evaporation, (ii) to increase friction, or (iii) to allow for application of a disinfectant to the surface of the gel.

19. The microwave measurement system of claim 16 wherein the gel is arranged with a layered structure, where each layer comprises a respective amount of ingredients or a respective salt concentration, thereby altering electromagnetic properties of the gel layer by layer

20. The microwave measurement system of claim 15 wherein the a shape of the gel is configured to conform to a part of the microwave measurement system to reduce coupling of microwave signals involving the part of the microwave system or to attenuate an interfering microwave signal generated by an interfering microwave transmitter received by the part of the microwave system.
